(19) United States
(12) Patent Application Publication
(10) Pub. No.: US 2011/0091853 A1
(43) Pub. Date: Apr. 21, 2011

(54) METHOD FOR SIMULATING A CATHETER GUIDANCE SYSTEM FOR CONTROL, DEVELOPMENT AND TRAINING APPLICATIONS

(75) Inventors: Yehoshua Shachar, Santa Monica, CA (US); Bruce Mark, Ojai, CA (US); David Johnson, West Hollywood, CA (US); Leslie Farkas, Ojai, CA (US); Steven Kim, New York, NY (US)

(73) Assignee: Magnetecs, Inc., Inglewood, CA (US)

(21) Appl. No.: 12/582,621

(22) Filed: Oct. 20, 2009

(51) Int. Cl. G09B 23/28; A61B 5/00; G09B 9/00; G05B 13/04

(52) U.S. Cl. 434/262; 600/424; 700/30

(57) ABSTRACT

A system is disclosed for incorporating a realistic simulated catheter or catheters within a catheter guidance and control system that operate from the same closed-loop position control feedback and geometric mapping data as the real position control system and are able to make contact with real and simulated datasets. These catheters may be operated in a pure simulation mode without interacting with real catheters and position control hardware, or may be used as control cursors to enhance the placement of catheter positioning targets. The catheter tip, which is focus of magnetic control, is realistically guided by the control system parameters, while the remainder of the catheter line is realistically constrained by the mapped chamber geometry and introducer sheath.

### BACKGROUND OF THE INVENTION

1. Field of the Invention

The invention relates to the systems and methods for guiding, steering and advancing an invasive medical device in a patient.

2. Description of the Related Art

In catheter guidance systems, control cursors are used to locate the desired position of the catheter tip. Unfortunately, prior art cursors do not bind the position and orientation to the achievable bounds of the system.

Blume et al, U.S. Pat. No. 6,014,580 describes a cursor for specifying the desired position of a catheter using a cross-hair type cursor. This is a simple pointing device which is capable of specifying a static location or locations within a workspace, but does not give a prediction of how the catheter will be situated with respect to the mapping data.

### SUMMARY

The system described herein solves these and other problems by incorporating a catheter guidance and imaging system simulated model into a catheter control system and applying it to the use of predictive guidance, dynamic response, and full simulation of surgical procedures.

In one embodiment, a catheter guidance simulation is used to generate a secondary simulated catheter which may be controlled by the operator as an interactive cursor that realistically predicts the motion and end disposition and orientation of a catheter, and which is then used as a desired position target for the closed-loop regulator to guide a real catheter to that disposition.

In one embodiment, a catheter line is graphically rendered relatively realistically on a display, and constrained by the mapped geometry, between the live catheter position and the live sheath position.

In one embodiment, a catheter line is rendered realistically, and constrained by the mapped geometry, between a simulated catheter position and a live sheath position, as to connect a catheter control cursor to the actual catheter entry location.

In one embodiment, a catheter guidance simulation is used to train operators in the use of a catheter guidance system in the absence of a patient or control hardware. The range of motion and expected stability of catheter placement may be assessed in the absence of guidance and control hardware.

In one embodiment, a catheter guidance simulation is used to approximate the performance of a closed-loop regulator before it is tested on position control hardware. The dynamic response and interaction with geometric models present real-world scenarios for assessing the stability of closed-loop position control.

### BRIEF DESCRIPTION OF THE DRAWINGS

FIG. 1 is a block diagram of the placement of a catheter guidance and control simulation within a catheter guidance control and imaging system.

FIG. 2A is a block diagram of the internal algorithms of the simulated catheter physics and obstacle detection module.

FIG. 2B is a block diagram detail of the data in and out of the catheter flexure kinematic module.

FIG. 2C is a block diagram detail of the derivation of the net torque and force on the catheter tip based on the difference in actual and desired position.

FIG. 2D is a block diagram detail of the obstacle collision detection calculation adding contact normal vectors to the data for the physics calculations.

FIG. 2E is a block diagram detail of the torque, force and collision data to increment the simulated catheter's position.

FIG. 3 is an illustration of one embodiment of the invention where the simulation is used to create a virtual catheter cursor.

FIG. 4A is an illustration of a simulated catheter in proximity to an obstacle within the mapping geometry.

FIG. 4B is an illustration of a simulated catheter touching an obstacle.

FIG. 4C is an illustration of a simulated catheter touching an obstacle and rotating.

FIG. 4D is an illustration of a simulated catheter touching and obstacle and being forced back and downwards.

FIG. 5 is a block diagram of the catheter guidance system used as a simulator without any live patient data or hardware control.

FIG. 6 is a block diagram of the placement of the physics simulation within the navigation and control system rendering software that is used to generate data for a realistic catheter line.

FIG. 7 is an illustration of a flexible catheter line being constrained by a geometric mesh.

FIG. 8 is an illustration of the elements used to simulate the flexible catheter line being constrained by a geometric mesh with the associated constraint vectors.

### DETAILED DESCRIPTION

In one embodiment, a predictive kinematic algorithm is used to help determine the movement of a catheter based on control parameters. The operator either manually steers the catheter through the use of a joystick or similar fixture, or places a cursor where they wish the catheter to go and a predictive kinematic algorithm is used by the closed-loop control system to guide the catheter to that location.

In one embodiment, the operational parameters are commonly determined through finite element analysis and control loop feedback calculations, which are useful for predicting the general kinematics of the mechanical apparatus and stability of the control loop. The operator's experience with the ergonomics and responsiveness of the system are later tested in the completed guidance system. A simulated model of the catheter guidance system and its response to a variety of operator inputs and dynamic models is not generally available.

In the graphical rendering of catheters within a mapping system, the catheter electrode locations are used to reconstruct the location and size of the catheter as it exists between the electrodes. Those portions of the catheter that are not in close proximity to an electrode are not generally rendered, as there is no reliable positional reference. Where the catheter has an associated introducer with positional electrodes, there is no attempt to graphically connect the catheter tip to its origin from within its introducer.

Training for use of catheter guidance systems can be done by the use of a human analog model which is placed within the system and used as a physical representative of the anatomical structures. These models contain either static or moveable structures to allow the operator to practice the targeting of tissue locations and navigating around obstacles with the catheter guidance system.

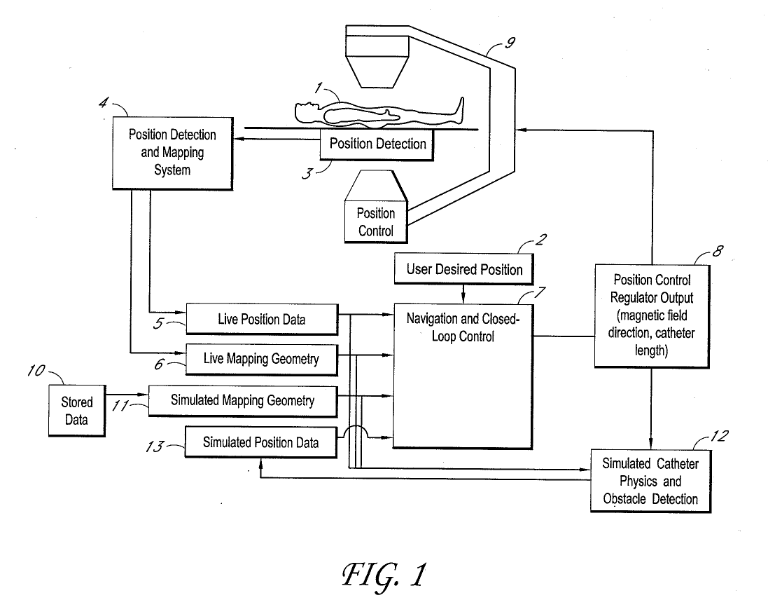

FIG. 1 is a block diagram of the placement of a catheter guidance and control simulation within a guidance control and imaging system. A patient 1 is placed in a guidance control and imaging system in a standard configuration. A position detection sensor system 3 is used by a position detection and mapping system 4 to continuously monitor the location and orientation of the tools within the patient. A guidance system's navigation and closed-loop control system 7 receives the live tool position data 5 from the position detection and mapping system 4, as well as the 3D geometry 6 created by the mapping process. The navigation and closed-loop control module 7 outputs a set of control parameters 8 to the position control system 9. These control parameters are the closed-loop control feedback that is used to synchronize the tool's actual position (AP) 5 with the operator's desired position (DP) 2. FIG. 1 further shows that simulated tool position data 13 and simulated mapping geometry 11 from stored computer data 10 can be used in parallel with live data in the navigation and closed-loop control system 7 by the use of a simulated catheter physics and obstacle detection computer algorithm 12. The input of this algorithm is the real and simulated tool position and mapping data, and the output of this algorithm is the simulated position data 13 based on the real navigation and closed-loop control system's position control regulator output 8.

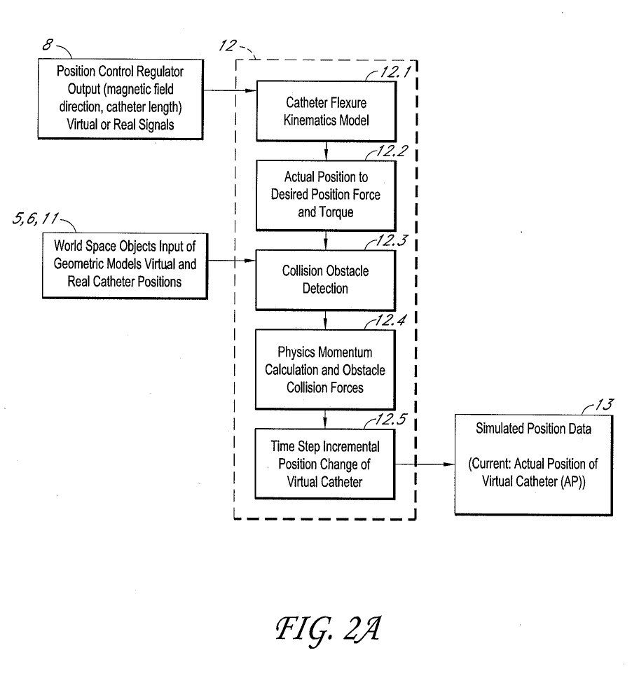

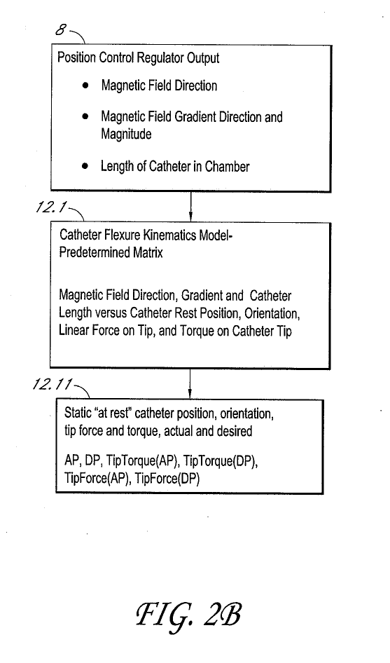

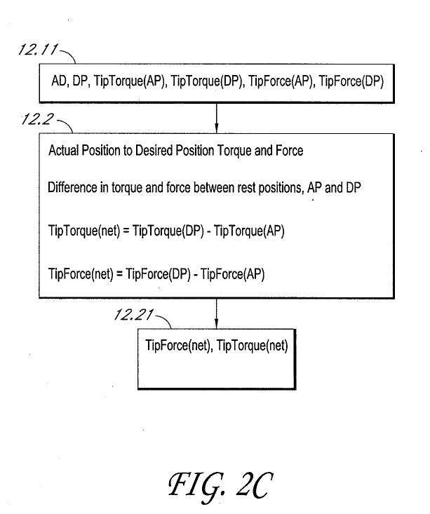

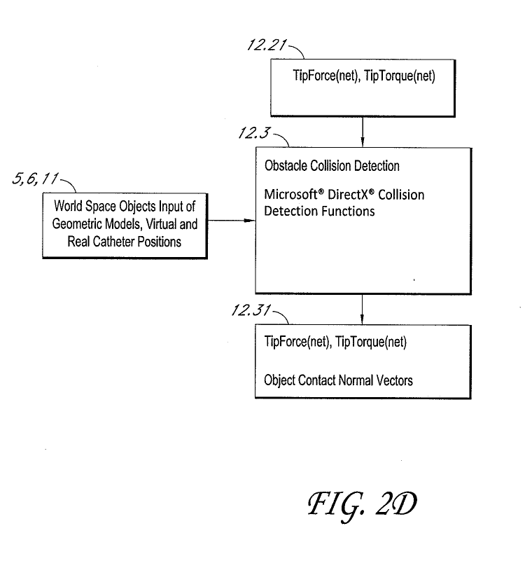

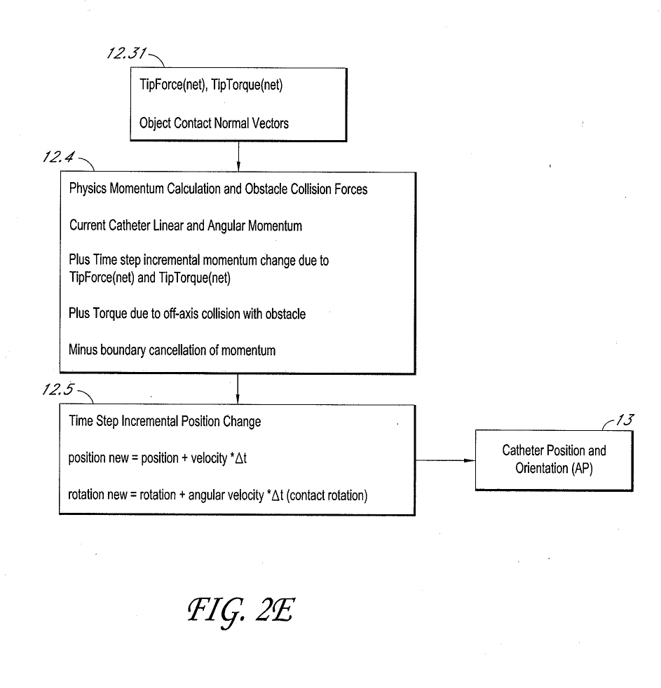

FIG. 2A is a block diagram of the internal algorithms of the simulated catheter physics and obstacle detection module 12. The closed-loop regulator's position control regulator output 8 is first used to calculate the rest position of the tool under the current regulator parameters. This is done with a predetermined model for catheter flexure kinematics 12.1 that relates the regulator parameters to the tool position and orientation at a rest state. The difference between the actual position of the tool and the kinematic rest position is used to calculate the force and torque on the simulated tool using the actual position to desired position force and torque algorithm 12.2. This actual position to desired position force and torque algorithm compiles the difference between the force and torque at the desired position and the force and torque at the current position, as specified by the predetermined kinematic model. Both real and simulated world space objects (5, 6 and 11) are used by the obstacle collision detection algorithm 12.3 to calculate the torques and forces that oppose the tool's motion. These forces and torques are then used by the physics momentum calculation and obstacle collision force algorithm 12.4 to adjust the instantaneous velocity and rotation of the tool. These dynamic properties are used by the time step incremental position change algorithm 12.5 to give a discrete position change of the tool based on the closed-loop time increments. The new simulated tool position 13 is output back to the guidance and imaging system.

FIG. 2B is a block diagram detail of the data in and out of the catheter flexure kinematic module. The position control regulator's output 8 passes the key regulation parameters to the kinematic model 12.1, including the magnetic field direction and magnetic field gradient and the length of the catheter. The kinematic model takes these parameters and outputs the ultimate static rest position and the physical forces on a catheter under the influence of those control system settings 12.1.1.

FIG. 2C is a block diagram detail of the derivation of the net torque and force on the catheter tip based on the difference in actual and desired position. The theoretical net forces 12.1.1 on the catheter are given by the kinematic model for both the current actual position of the catheter (AP) and the desired static rest position (DP) of the catheter under the current control forces. These are subtracted by the actual position to desired position torque and force module 12.2 to give the net torque and force on the catheter 12.2.1 which is passed to the physics momentum calculation module 12.4 (see FIG. 2E).

FIG. 2D is a block diagram detail of the obstacle collision detection calculation adding contact normal vectors to the data for the physics calculations. Industry standard routines 12.3 are used to detect collisions between the world space objects (5, 6 and 7) and the catheter which output the contact locations and contact normal vectors 12.3.1 to the physics momentum calculation module 12.4. The net torque and force 12.2.1 is also passed on from the previous module.

FIG. 2E is a block diagram detail of the torque, force and collision data to increment the simulated catheter's position. The net forces on the catheter tip from the control system and object collisions 12.3.1 are used by the physics momentum calculation and obstacle collision forces algorithm 12.4 to incrementally increase the linear and angular velocity of the catheter tip. Obstacle collisions are used to terminate the velocity in specific directions, and to impart an additional angular velocity due to off-axis collisions. The net linear and angular velocity are stored and used by the time step incremental position change algorithm 12.5 to incrementally change the catheter actual position and orientation (AP) 13.

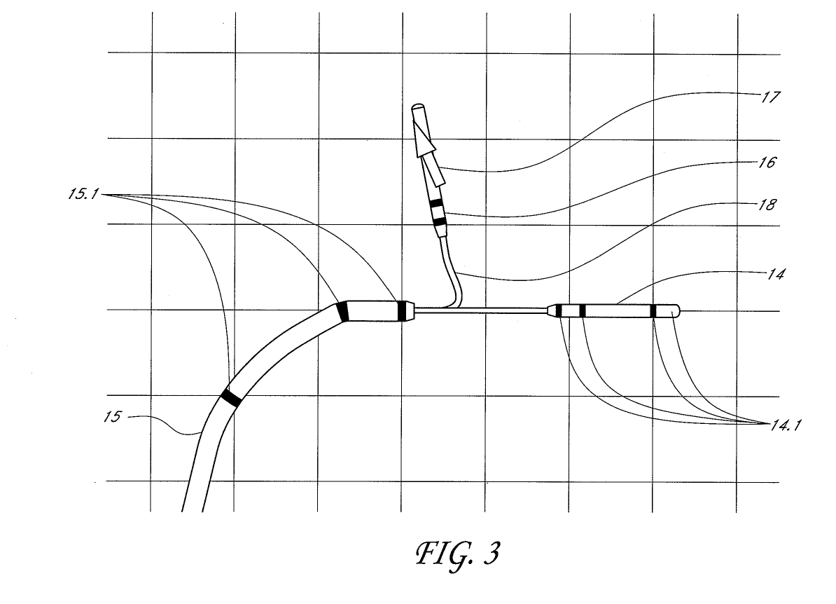

FIG. 3 is an illustration of one embodiment where the simulation is used to create a virtual catheter cursor. This provides the operator with an interactive cursor which gives a realistic view of the range of catheter motion and potential interaction with the geometric mapping data. The live position of the physical catheter 14 and the physical introducer sheath 15, based on their electrode positions (14.1, 15.1), are displayed from the position detection and mapping system data 5. The catheter and introducer sheath are graphically rendered to depict the catheter 14 emerging from the sheath 15. The simulated regulator output, indicated by the magnetic control arrow 17 is based on the operator input and is used to calculate and render a simulated catheter 16 which is also shown to emerge from the live physical sheath 15. The flexible shaft of the simulated catheter 18 behaves as a physical catheter would, which gives the operator a visual reference of the amount of catheter in the chamber and the probable disposition of the catheter line about the chamber.

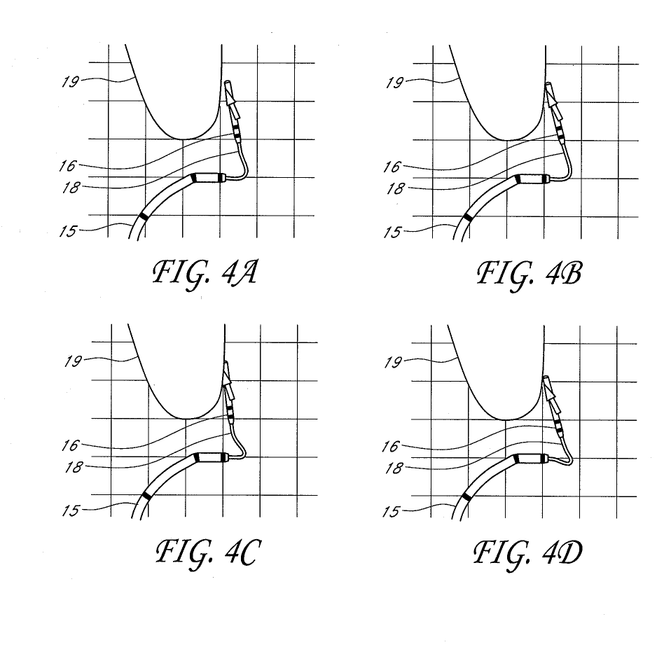

FIG. 4A is an illustration of a simulated catheter in proximity to an obstacle within the mapping geometry. The obstacle 19 may be either from the real-world mapping system data or a simulated object. The catheter 16 emerges from the sheath 15, and the catheter's flexible section 18 is depicted as a smooth tangent curve.

FIG. 4B is an illustration of a simulated catheter touching an obstacle. The simulated catheter 14 is just making contact with the obstacle 19, so the catheter line 18 is depicted as a smooth tangent curve.

FIG. 4C is an illustration of a simulated catheter touching an obstacle and rotating. The simulated catheter 14 is making strong contact with the tip, causing the tip to rotate. The catheter line is depicted as having a non-tangent “s” curve due to the applied torque to the tip.

FIG. 4D is an illustration of a simulated catheter touching an obstacle and being forced back and downwards. The catheter line is depicted as having a relatively sharp downward pinch, indicating that the catheter is being pushed back and downward.

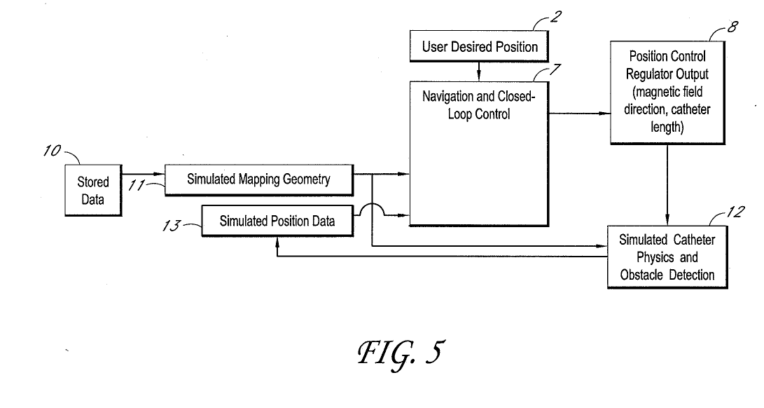

FIG. 5 is a block diagram of the catheter guidance system used as a simulator without any live patient data or hardware control. The user's desired position input 2 is used by the navigation and closed-loop control module 7 to send position control regulator output 8 to the simulated catheter physics and obstacle detection engine 12. The simulated position data 13 is based on the user's desired input 2 and the simulated mapping geometry 11 from stored data 10. In one embodiment of the invention, the simulator may be used to develop and tune the closed-loop regulation of the navigation and closed-loop control module 7 as to give an approximation of the expected performance before the control loop is used with the sensitive hardware. In another embodiment of the invention, the simulator can be used to train operators without the patient and control hardware.

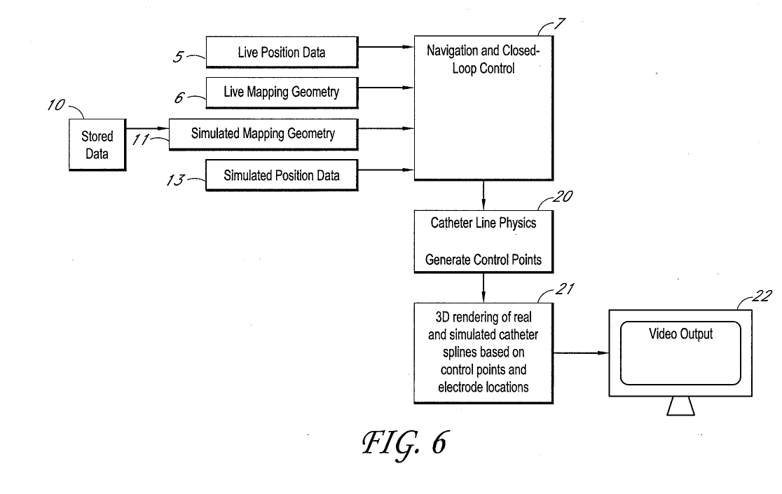

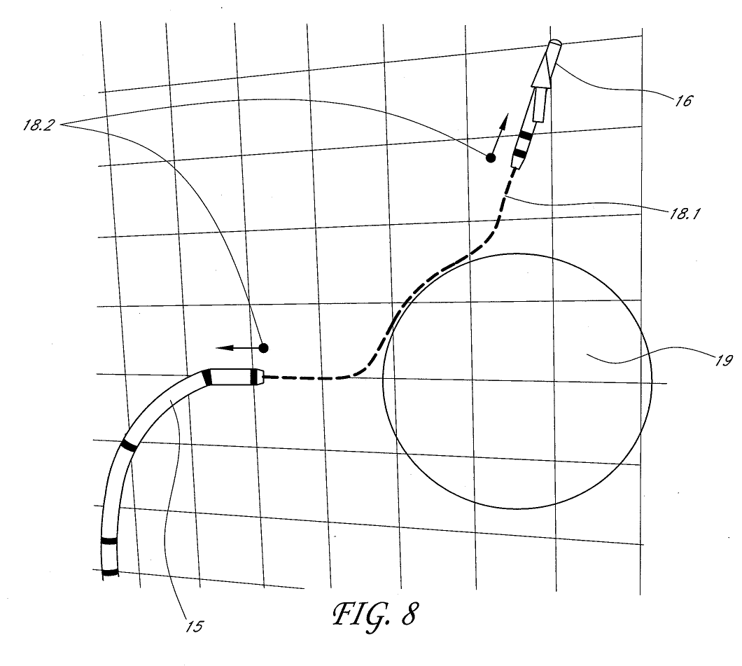

FIG. 6 is a block diagram of the placement of the physics simulation within the navigation and control system rendering software that is used to generate data for a realistic catheter line. In one embodiment, a Runge-Kutta Ordinary Differential Equation based physics simulation 20, such as, for example, the open source Tokamak Game Physics SDK, can be used to generate additional location data needed to render the line between the sheath 15 and the catheter tip 16. The navigation and closed-loop control module 7 passes the live position data 5 for the catheters, live mapping data 6, simulated mapping geometry 11 from stored data files 10 and simulated catheter position data 13 through to the 3D rendering engine 21 and the catheter line physics simulation 20. The rendering engine renders the catheters by their electrode location data (5, 13). The catheter line physics calculations 20 insert the additional catheter line position data before the catheters are rendered 21 and displayed 22. These control points 18.1 (see FIG. 8) are used by the rendering engine 21 in a similar fashion as the electrode location data to render the geometric spline meshes.

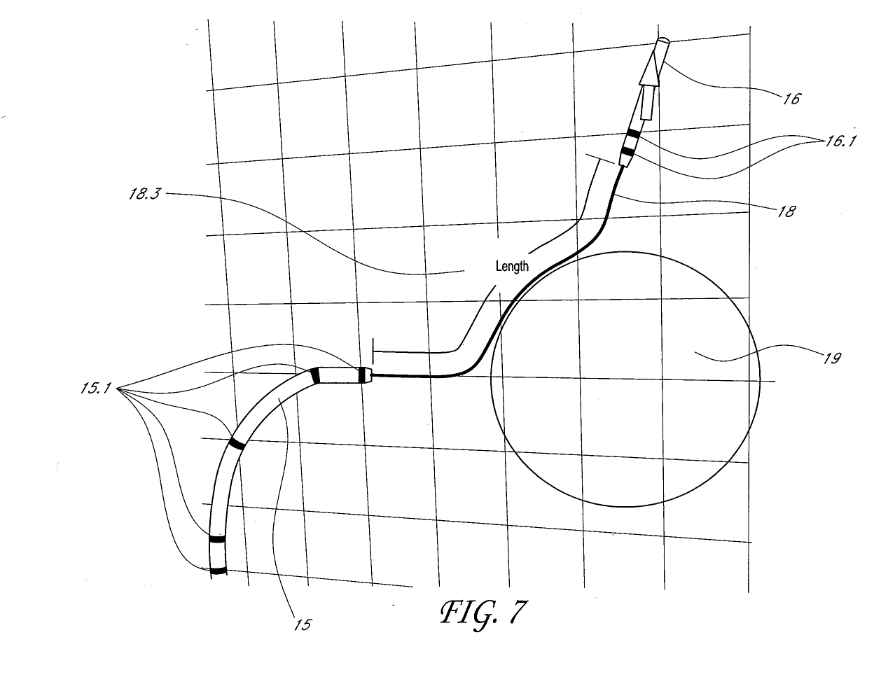

FIG. 7 is an illustration of a flexible catheter line being constrained by a geometric mesh. The catheter line 18 emerges from the sheath 15 and connects to the catheter tip 16. The catheter tip 16 and sheath 15 locations are defined and rendered according to their electrode positions, 15.1 and 16.1. The catheter line 18 is rendered according to the control points 18.1 (see FIG. 8) generated by a simple physics simulation 20, such as the open source Tokamak Game Physics SDK, which generates a realistic interaction between the control points and all real and simulated geometric objects (see FIG. 1, assemblies 5, 6, 11 and 13) including the spherical mesh obstacle 19. The end constraints 18.2 (see FIG. 8) are the catheter tip 16 and sheath 15 locations. The length of the catheter line 18.3 is passed to the physics engine by the navigation and closed-loop control module 7.

FIG. 8 is an illustration of the elements used to simulate the flexible catheter line being constrained by a geometric mesh with the associated constraint vectors. The catheter line that emerges from the sheath 15 and connects to the catheter tip 16 has been represented as a series of control points 18.1, each with its own constraints. The simple physics simulation creates a realistic interaction between the elements and all of the live and simulated geometric objects (5, 6, 11 and 13). Where the flexible catheter line segment 18 makes contact with the chamber geometry or obstacle 19, the line conforms to the shape and does not pass through the wall, behaving like a physical catheter. The acquired location of the physical or simulated catheter tip 16 and the physical sheath 15 become the end constraints 18.2 for the flexible segment control points 18.1. The length of the segment within the chamber 18.3, the geometric models (6, 11) and the end constraints are sent to the physics engine 20 to generate the location of the catheter line control points 18.1.

It is to be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but can be used alone or combined in other combinations. The excision of any disclosed element of the invention is explicitly contemplated as within the scope of the invention.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense, an equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a sub combination or variation of a sub combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the invention is limited only by the claims.

What is claimed is:

1. A catheter guidance and control simulation system, comprising:
a position detection and mapping system that receives sensor data from a sensor that detects a catheter, said position detection and mapping system producing catheter position data;
a closed-loop control system that receives the catheter position data and 3D geometry created by a mapping process, said closed-loop control system providing output control parameters a position control system that controls a position of the catheter; and
a simulation system that receives said catheter position data, said 3D geometry, and said output control parameters and computes a predicted catheter position, said predicted catheter position provided to said closed-loop control system, said closed-loop control system adjusting said output control parameters in response to said predicted catheter position.

2. The system of claim 1, further comprising a display that displays an actual catheter position using said catheter position data and a simulated catheter position using said predicted catheter position.

3. The system of claim 1, wherein said simulation system comprises a Runge-Kutta Ordinary Differential Equation based physics engine to simulate the geometry and dynamics of the catheter within a three dimensional manifold.

4. A simulation and training system, comprising:
a user input control;
a control module that receives user input data from said user input control and produces control outputs; and
a simulation system that receives said user input data, mapping data, and said control outputs and computes simulated position data of a catheter according to a physics model of a catheter in a body cavity and controlled by a system of one or more electromagnets, said simulated position data provided to said control module, said control module configured to use said simulated position data as feedback of an actual catheter position and to compute said control outputs using closed-loop feedback.
